(19) United States
(12) Patent Application Publication
(10) Pub. No.: US 2024/0024298 A1
(43) Pub. Date: Jan. 25, 2024

(54) COMPOSITIONS AND METHODS FOR THE TREATMENT OF HPV-ASSOCIATED DISEASE STATES

(71) Applicant: Children's Hospital Medical Center, Cincinnati, OH (US)

(72) Inventors: Susanne Wells, Cincinnati, OH (US); Taylor Lange, Cincinnati, OH (US); Kanimozhi Vairamani, Cincinnati, OH (US)

(21) Appl. No.: 18/224,632

(22) Filed: Jul. 21, 2023

Related U.S. Application Data
(60) Provisional application No. 63/391,389, filed on Jul. 22, 2022.

(51) Int. Cl. A61K 31/437; C12Q 1/6883

(52) U.S. Cl. CPC A61K 31/437; C12Q 1/6883

(57) ABSTRACT

Disclosed are methods and compositions for treating a human papillomavirus (HPV)-related disease or cancer in an individual in need thereof. The method may comprise administering a glucosylceramide synthase (GCS) inhibitor to the individual, for example, miglustat, eligustat, venglustat, T-036, and combinations thereof. The disclosed methods and compositions may be used to treat cancers such as cervical cancer, oropharyngeal cancer, vulvar cancer, anal cancer, penile cancer, vaginal cancer, rectal cancer, squamous cell carcinoma, adenocarcinoma, head and neck cancer, or skin or mucosal lesions such as a common wart, a genital wart, a respiratory wart, a plantar wart, a subungual wart, a periungual wart, a flat wart, and combinations thereof.

### CROSS-REFERENCE TO RELATED APPLICATIONS

This application claims priority to and benefit of U.S. Provisional Application Ser. No. 63/391,389, filed Jul. 22, 2022, the contents of which are incorporated in their entirety for all purposes.

### BACKGROUND

Human papillomavirus, or HPV, is a virus that infects many people. 80 million Americans are currently infected and 14 million adolescents are newly infected each year. High-risk (HR) HPV infections can lead to the development of squamous cell carcinoma (SCCs).

While most HPV infections are subclinical and will cause no physical symptoms, in some people infections may cause growths known as papillomas, and may even cause cancers of the cervix, vulva, vagina, penis, oropharynx and anus. In particular, HPV16 and HPV18 are known to cause around 70% of cervical cancer cases. HPV is a known cause of cervical cancers, as well as as well as head and neck cancers, vulvar, vaginal, penile, oropharyngeal, anal, perianal, and rectal cancers. High-risk oncogenic HPV types are able to integrate into the host DNA and express genes such as HPV E6 and E7. It is thought that the E6 and E7 oncoproteins inactivate p53 and pRB tumor suppressors, implicating HPV in the development of cancer Although most HPV infections are asymptomatic and clear spontaneously, persistent infections with one of the oncogenic HPV types can progress to precancer or cancer. Other HPV-associated diseases can include common warts, plantar warts, flat warts, anogenital warts, anal lesions, epidermodysplasia, focal epithelial hyperplasia, mouth papillomas, verrucous cysts, laryngeal papillomatosis, squamous intraepithelial lesions (SILs), cervical intraepithelial neoplasia (CIN), vulvar intraepithelial neoplasia (VIN) and vaginal intraepithelial neoplasia (VAIN). (U.S. 2021003870).

While vaccines are available, current iterations have only poor uptake and there are currently no treatments for HPV infection. Thus, there is a need for improved methods of treating HPV-related disease states.

### BRIEF SUMMARY

Disclosed are methods and compositions for treating a human papillomavirus (HPV)-related disease or cancer in an individual in need thereof. The method may comprise administering a glucosylceramide synthase (GCS) inhibitor to the individual, for example, miglustat, eligustat, venglustat, T-036, and combinations thereof. The disclosed methods and compositions may be used to treat cancers such as cervical cancer, oropharyngeal cancer, vulvar cancer, anal cancer, penile cancer, vaginal cancer, rectal cancer, squamous cell carcinoma, adenocarcinoma, head and neck cancer, or skin or mucosal lesions such as a common wart, a genital wart, a respiratory wart, a plantar wart, a subungual wart, a periungual wart, a flat wart, and combinations thereof.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 shows HPV− and HPV+ keratinocytes grown into organotypic epithelial rafts over the course of 14 days. On raft day 14, the tissues were harvested and lysed with RIPA buffer to extract protein for western blotting. (A) representative western blot is shown demonstrating that HPV+ rafts have overall more O-Glacylated protein (O-GlcNAc) than HPV− rafts with no change in protein expression of OGT, the enzyme responsible for 0-GlcNAcylation. Beta-actin was used to show equal protein loading. (B) HPV− and HPV+ rafts were treated with increasing doses of OSMI-1, an inhibitor of OGT activity, from day 8 to day 14 of the raft culturing protocol. EdU, a thymidine analog that incorporates into DNA during DNA synthesis, was added to the media on day 13. 24 h later, on raft day 14, tissues were fixed in PFA for paraffin-embedding, sectioning, and immunofluorescence (IF) staining. Representative IF images are shown with nuclei stained with DAPI (blue), and proliferating cells visualized with click chemistry to detect EdU incorporation (pink). Quantification of the number of EdU+ cells per field is shown to the right. Vehicle treated HPV+ rafts had significantly more proliferating cells than the HPV− controls. Proliferation was significantly decreased in the HPV+ rafts at the 6.25 uM OSMI-1 dose with no effect on proliferation in the HPV− rafts. The highest dose tested, 25 uM, decreased proliferation in both the HPV− and HPV+ rafts. 2-way ANOVA was used to determine significance. Representative images shown. n=3.

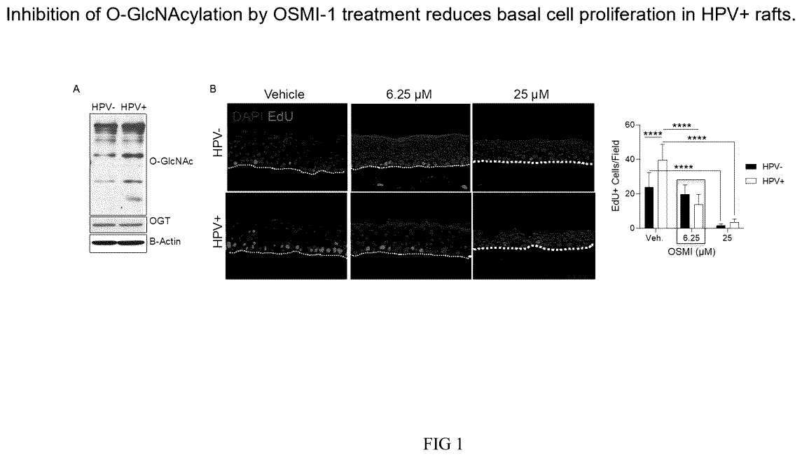

FIG. 2 illustrates that HPV16 infection upregulates ganglioside production. (A) Schematic representation of the first steps to the ganglioside synthesis pathway and the ganglioside species produced during each step. Enzymes that were upregulated or downregulated in the bulk RNA sequencing data are labeled with red and blue colored text respectively. The rate limiting step of the pathway, the conversion of ceramide to GlcCer by the enzyme GCS, can be specifically inhibited with clinically used FDA approved inhibitors such as Miglustat and Eliglustat. (B) Mass spectrometry results showing that HPV+ rafts have upregulated levels of gangliosides GM3, GM1, GM2, and GD1a as compared to HPV− rafts, which correlates with the enzyme gene expression patterns depicted in the schematic (A). n=5.

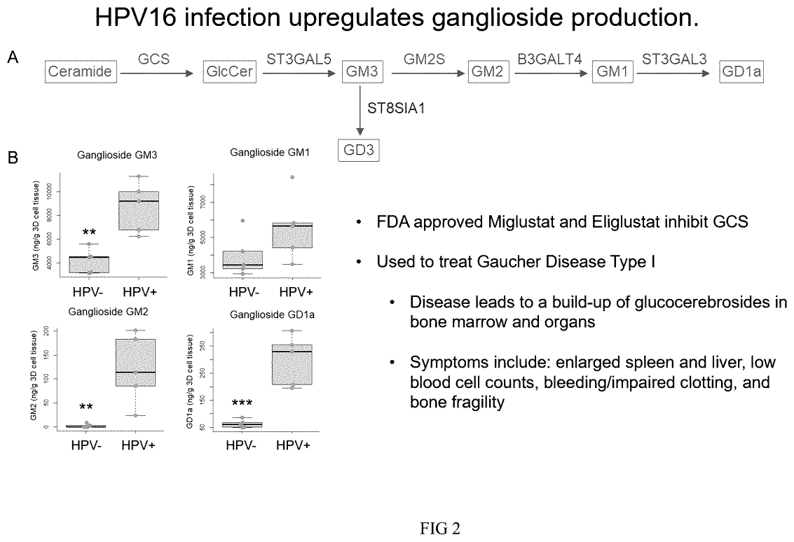

FIG. 3 (A) HPV+ and HPV− keratinocytes were seeded in round-bottom plates to form 3D spheroids (Day 0). 48 h after plating the cells (Day 2), increasing doses of Eliglustat were added to the media. 48 h after adding the drug treatment (Day 4), spheroids were imaged with brightfield microscopy and images quantified for sphere area. n=4. (B) Quantification of sphere area on culture day 4 after treatment with increasing doses of Eliglustat for 48 h. The area of HPV+ spheres decreased concomitantly with increasing Eliglustat dose with the highest dose tested, 25 uM, significantly reduce HPV+ sphere area to that of HPV− spheres. n=4.

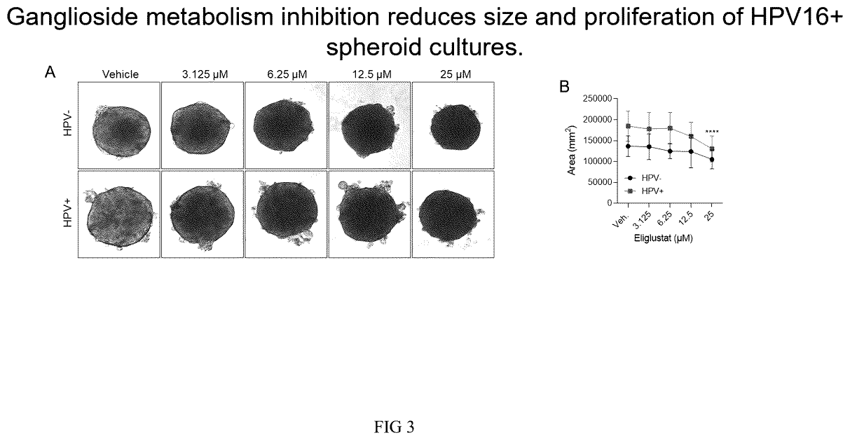

FIG. 4 shows that HPV− and HPV+ rafts were treated with increasing doses of Eliglustat, an inhibitor of GCS activity, from day 8 to day 14 of the raft culturing protocol, and EdU added on day 13. Representative IF images are shown with nuclei stained with DAPI (blue), proliferating cells visualized with click chemistry to detect EdU incorporation (white), basal cell layer keratinocytes stained with collagen 17A (COL17A; green) and differentiated layer keratinocytes stained with keratin 10 (K10; red). Quantifications of the total (top graph) and suprabasal (lower graph) number of EdU+ cells per field are shown to the right. As expected, vehicle treated HPV+ rafts had significantly more proliferating cells than the HPV− controls. Proliferation was significantly decreased with increasing dose in the Eliglustat treated HPV+ rafts with no significant effect on proliferation in the HPV− rafts. 2-way ANOVA was used to determine significance. Representative images shown. n=3.

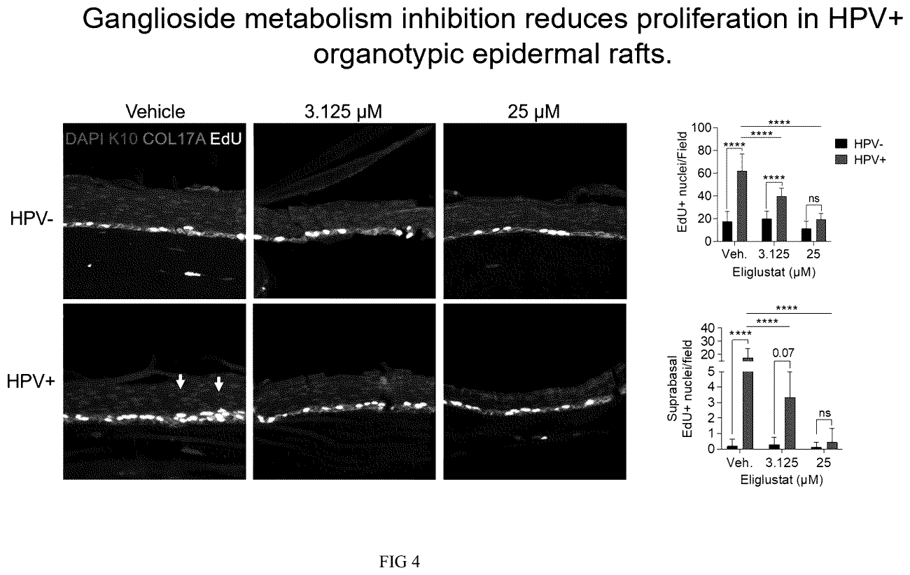

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting. The methods may comprise, consist of, or consist essentially of the elements of the compositions and/or methods as described herein, as well as any additional or optional element described herein or otherwise useful in the treatment of HPV-related diseases using a GCS inhibitor.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

The active agent may form salts, which are also within the scope of the preferred embodiments. Reference to a compound of the active agent herein is understood to include reference to salts thereof, unless otherwise indicated. The term “salt(s)”, as employed herein, denotes acidic and/or basic salts formed with inorganic and/or organic acids and bases. In addition, when an active agent contains both a basic moiety, such as, but not limited to an amine or a pyridine or imidazole ring, and an acidic moiety, such as, but not limited to a carboxylic acid, zwitterions (“inner salts”) may be formed and are included within the term “salt(s)” as used herein. Pharmaceutically acceptable (e.g., non-toxic, physiologically acceptable) salts are preferred, although other salts are also useful, e.g., in isolation or purification steps, which may be employed during preparation. Salts of the compounds of the active agent may be formed, for example, by reacting a compound of the active agent with an amount of acid or base, such as an equivalent amount, in a medium such as one in which the salt precipitates or in an aqueous medium followed by lyophilization. When the compounds are in the forms of salts, they may comprise pharmaceutically acceptable salts. Such salts may include pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Examples of metal salts include lithium, sodium, potassium, magnesium salts and the like. Examples of ammonium and alkylated ammonium salts include ammonium, methylammonium, dimethylammonium, tri methyl ammonium, ethyl ammonium, hydroxy ethyl ammonium, diethylammonium, butylammonium, tetramethylammonium salts and the like. Examples of organic bases include lysine, arginine, guanidine, diethanolamine, choline and the like.

“Sequence identity” as used herein indicates a nucleic acid sequence that has the same nucleic acid sequence as a reference sequence, or has a specified percentage of nucleotides that are the same at the corresponding location within a reference sequence when the two sequences are optimally aligned. For example a nucleic acid sequence may have at least 50%, 60%, 70%, 75%, 80%, 85%, 90%, 95%, 96%, 97%, 98%, 99%, or 100% identity to the reference nucleic acid sequence. The length of comparison sequences will generally be at least 5 contiguous nucleotides, preferably at least 10, 11, 12, 13, 14, 15, 16, 17, 18, 19, 20, 21, 22, 23, 24, or 25 contiguous nucleotides, and most preferably the full length nucleotide sequence. Sequence identity may be measured using sequence analysis software on the default setting (e.g., Sequence Analysis Software Package of the Genetics Computer Group, University of Wisconsin Biotechnology Center, 1710 University Avenue, Madison, Wis. 53705). Such software may match similar sequences by assigning degrees of homology to various substitutions, deletions, and other modifications.

As used herein, “treatment” encompasses administering the disclosed compositions for obtaining a beneficial or desired clinical result. “Treatment” as used herein, covers any administration or application of a therapeutic for disease in a mammal, including a human. Beneficial or desired clinical results include, but are not limited to, any one or more of: alleviation of one or more symptoms, diminishment of extent of disease, preventing or delaying spread (e.g., metastasis, for example metastasis to the lung or to the lymph node) of disease, preventing or delaying recurrence or emergence of disease, delay or slowing of disease progression, amelioration of the disease state, inhibiting or slowing the disease or progression of the disease, arresting disease development, and facilitating partial or total remission. Also encompassed by “treatment” is a reduction of pathological consequence of a proliferative disease. The instant disclosure contemplates any one or more of these aspects of treatment. In the context of cancer, the term “treating” includes any or all of: inhibiting growth of cancer cells, inhibiting replication of cancer cells, lessening of overall tumor burden and ameliorating one or more symptoms associated with the disease.

The instant disclosure relates to compositions and methods for the treatment of a human papillomavirus (HPV)-related disease state in an individual in need thereof, comprising administering a glucosylceramide synthase (GCS) inhibitor to the individual. Glucosylceramide synthase (GCS), also known as UDP-glucose ceramide synthase, is the enzyme which catalyzes the initial glycosylation step of ceramide to form glucosylceramide. In one aspect, the disease state is a cancer. The cancer may be any cancer that is determined to be caused, or substantially caused or accelerated by HPV, and may include, for example, a cancer selected from cervical cancer, oropharyngeal cancer, vulvar cancer, anal cancer, penile cancer, vaginal cancer, rectal cancer, squamous cell carcinoma, adenocarcinoma, and head and neck cancer.

In one aspect, the HPV-associated disease may be a skin or mucosal lesion caused by, substantially caused by, or accelerated by HPV, and may include, for example, a common wart, a genital wart, a respiratory wart, a plantar wart, a subungual wart, a periungual wart, a flat wart, and combinations thereof.

HPV types are classified into fifteen “high risk types” (HPV 16, 18, 31, 33, 35, 39, 45, 51, 52, 56, 58, 59, 68, 73, and 82) and three “probable high risk types” (HPV 26, 53, and 66), which together are known to manifest as low and high grade cervical changes and cancers, as well as other anogential cancers such as vulval, vaginal, penile, anal, and perianal cancer, as well as head and neck cancers. Recently, the association of high risk types HPV 16 and 18 with breast cancer was also described. Eleven HPV types classified as “low risk types” (HPV 6, 11, 42, 43, 44, 54, 61, 70, 72, and 81) are known to manifest as benign low-grade cervical changes, genital warts and recurrent respiratory papillomatosis. Cutaneous HPV types 5, 8, and 92 are associated with skin cancer. In some HPV-associated cancers, the immune system is depressed and correspondingly, the antitumor response is significantly impaired. See US20210038709. In one aspect, the HPV may be a high-risk type HPV. For example, the high-risk HPV type may be selected from 16, 18, 31, 33, 34, 35, 39, 45, 51, 52, 56, 58, 59, 66, 68, and combinations thereof. In one aspect, the HPV type may be one or both of type 16 and type 18. In one aspect, the HPV type may be a low-risk type HPV. For example, the low-risk HPV type may be selected from 6, 11, 42, 43, and 44.

The GCS inhibitor administered to the individual in need thereof may be any known or suspected GCS inhibitor. For example, in one aspect, the GCS inhibitor may be selected from miglustat, eligustat, venglustat, T-036, and combinations thereof, T-36 having the following structure, available from MedKoo Biosciences:

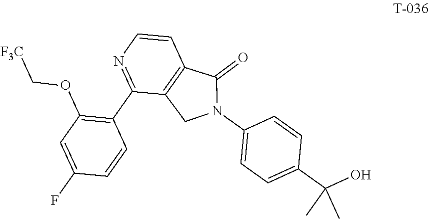 [SMILES: CC(C)(O)c1ccc(N2Cc3c(ccnc3-c3ccc(F)cc3OCC(F)(F)F)C2=O)cc1]

Eliglustat is a glucosylceramide (GlcCer) analogue that acts as an inhibitor of glucosylceramide synthase (GCS), a Golgi complex enzyme that catalyzes the formation of glucosylceramide from ceramide and UDP-glucose and is the first step in the formation of glucocerebroside-based glycosphingolipids. Miglustat (Zavesca), is an iminoglucose GCS inhibitor. It is an N-alkylated iminosugar and acts as a reversible competitive inhibitor of GCS, binding in the enzyme's active site. Eliglustat is also a GCS inhibitor, and it is an analogue of the ceremide. Eliglustat and venglustat are described in ClinicalTrials.gov identifier NCT00891202 ClinicalTrials.gov identifier NCT02906020.

In one aspect, the methods may include detecting an abnormal cytology in the individual, prior to treating the individual with a GCS inhibitor. In a further aspect, the methods may include detecting the presence of HPV in a biological sample obtained from an individual. The detecting of HPV may be carried out prior to, during, or following the administering of a GCS inhibitor. Detection of HPV may be carried out via known methods in the art. Such methods may include detecting a nucleic acid or protein that indicates the presence of HPV in a biological sample from the individual. An exemplary method is described in, for example, US application serial no. US20230002839, which describes primer sets for amplifying the nucleotide sequence of the L2 gene of human papillomavirus type 16 or L1 gene of human papillomavirus type 18. In one aspect, the protein that is detected may be p16. In one aspect, the p16 detected may be determined to be overexpressed as compared to a baseline or control level of p16. In one aspect, the detecting comprises detecting a nucleic acid which indicates the presence of one or both of an E6 gene and an E7 gene, such methods being known in the art and described in, for example, Yao Y L et al, Human papillomavirus (HPV) E6/E7 mRNA detection in cervical exfoliated cells: a potential triage for HPV-positive women. J Zhejiang Univ Sci B. 2017 Mar.; 18(3):256-262. doi: 10.1631/jzus.B1600288. PMID: 28271661; PMCID: PMC5369250. The sample in which detection is carried out may be determined by one of ordinary skill the art. For example, in one aspect, the biological sample may be a tissue sample obtained from the individual. The biological sample taken from a tissue of the individual selected from an area in which HPV infection is suspected, for example, a lymph node or lesion in the head, neck, or anogenital tract, or any tissue in which an accumulation of HPV virus is suspected.

The administration of the GCS inhibitor may be prior to a diagnosis of an HPV-related cancer, or following a diagnosis of an HPV-related cancer. In one aspect, the individual may be diagnosed with an HPV infection at the time, or prior to administration of the GCS inhibitor. For example, the individual may be diagnosed as having an HPV infection for a period of time of more than one week, more than two weeks, more than three weeks, more than four weeks, more than five weeks, more than six weeks, more than seven weeks, more than two months, more than three months, more than four months, more than five months, more than six months, more than seven months, more than eight months, more than nine months, more than ten months, more than 11 months, more than 12 months, 18 months, or more than two years, or more than three years, or more than four years, or more than five years.

The administration of the GCS inhibitor may be for a period of time of at least one day, or at least two days, or at least three days, or at least four days, or at least five days, or at least six days, or at least seven days, or at least two weeks, or at least three weeks, or at least four weeks, or at least two months, or at least three months, or at least four months, or at least or at least five months, or at least six months, or up to a year or more. In one aspect, the GCS inhibitor is administered until the HPV or an indicator nucleic acid or protein thereof, is no longer detectable in a biological sample obtained from the individual.

The route of administration may vary, and may include more than one route. For example, routes of administration may include, for example, oral (such as via tablets, capsules, liquids, and powders), parenteral including intravenous, intramuscular (IM), subcutaneous (SC), intradermal (ID), intrathecal, or combinations thereof, topical (including via creams, ointments, gels, patches, nasal sprays and the like), inhalation, rectal, vaginal, transdermal (such as via skin patches, allowing for continuous absorption over an extended period), and intra-tumor, such as via injection directly to a tumor. In one aspect, at least two, or at least three, or at least four or more routes of administration may be used together or sequentially to treat an individual. In one aspect, bott a topical and systemic route may be used. In one aspect, the HPV-related cancer is penile, anal, vulvar, cervical, and the administration is topical. In one aspect, the HPV-related cancer is head and neck cancer, and the administration is via intra-tumor injection. In one aspect, the compositions are formulated in a medicament that is suitable for topical application, such as a formulation suitable for topical delivery of the active ingredients to the cervix (e.g. as a gel, cream, soft capsule, or pessary) for preventing the development of, or treating cervical cancer (e.g. caused by high-risk types of HPV such as HPV16), or a skin lesion. The compositions may be topically applied to the cervix of women with a viral infection of the cervix with a view to treating the viral infection and thereby preventing the development of a cancer at a future date. The compositions may be used to prevent or treat cancer as a monotherapy (i.e. use of the two inhibitors alone) or in combination with other compounds or treatments used in cancer therapy (e.g. chemotherapeutic agents, radiotherapy). In one aspect, the HPV-related cancer is head and neck cancer, and said administration is systemic.

The exact formulation, route of administration, and dosage is determined by an individual physician in view of the diagnosed condition or disease and the individuals response to the GCS inhibitor. Dosage amount and interval can be adjusted individually to provide levels of a GCS inhibitor sufficient to maintain therapeutic effects. The effective amount for use in therapy may vary with the nature of the condition being treated, the length of time that activity is desired, and the age and the condition of the patient, and ultimately may be determined by the attending physician. Dosage amounts and intervals can be adjusted individually to provide plasma levels of the GCS inhibitor sufficient to maintain the desired therapeutic effects. The desired dose can be administered in a single dose, or as multiple doses administered at appropriate intervals, for example as one, two, three, four or more doses per day. The GCS inhibitor may be administered in an amount of about 0.005 to about 500 milligrams per dose, about 0.05 to about 250 milligrams per dose, or about 0.5 to about 100 milligrams per dose. For example, the GCS inhibitor can be administered, per dose, in an amount of about 0.005, 0.5, 5, 10, 20, 30, 40, 50, 60, 70, 75, 80, 85, 90, 95, 100, 125, 150, 175, 200, 225, 250, 275, 300, 325, 350, 375, 400, 425, 450, or 500 milligrams, including all doses between 0.005 and 500 milligrams. composition containing the same, can be from about 1 ng/kg to about 200 mg/kg, about 1 μg/kg to about 100 mg/kg, or about 1 mg/kg to about 50 mg/kg. The dosage of a composition can be at any dosage including, but not limited to, about 1 μg/kg. The dosage of a composition may be at any dosage including, but not limited to, about 1 μg/kg, 10 μg/kg, 25 μg/kg, 50 μg/kg, 75 μg/kg, 100 μg/kg, 125 μg/kg, 150 μg/kg, 175 μg/kg, 200 μg/kg, 225 μg/kg, 250 μg/kg, 275 μg/kg, 300 μg/kg, 325 μg/kg, 350 μg/kg, 375 μg/kg, 400 μg/kg, 425 μg/kg, 450 μg/kg, 475 μg/kg, 500 μg/kg, 525 μg/kg, 550 μg/kg, 575 μg/kg, 600 μg/kg, 625 μg/kg, 650 μg/kg, 675 μg/kg, 700 μg/kg, 725 μg/kg, 750 μg/kg, 775 μg/kg, 800 μg/kg, 825 μg/kg, 850 μg/kg, 875 μg/kg, 900 μg/kg, 925 μg/kg, 950 μg/kg, 975 μg/kg, 1 mg/kg, 5 mg/kg, 10 mg/kg, mg/kg, 20 mg/kg, 25 mg/kg, 30 mg/kg, 35 mg/kg, 40 mg/kg, 45 mg/kg, 50 mg/kg, 60 mg/kg, mg/kg, 80 mg/kg, 90 mg/kg, 100 mg/kg, 125 mg/kg, 150 mg/kg, 175 mg/kg, or 200 mg/kg. The above dosages are exemplary and higher or lower dosages may be merited and are within the scope of the invention. The physician may determine the actual dosing regimen that is most suitable for an individual patient, which can vary with the age, weight, and response of the particular patient.

The medicaments used according to the invention may take a number of different forms depending, in particular, on the manner in which the medicament is to be applied topically. Thus, for example, the medicament may be in the form of a powder, tablet, capsule, liquid, ointment, cream, gel, hydrogel, ovule, suppository, aerosol, spray, micelle, liposome or any other suitable form that may be administered to a person or animal. It will be appreciated that the vehicle of the medicament of the invention should be one which is well tolerated by the subject to whom it is given and enables delivery of the inhibitors to the effected or target site.

In some aspects, the compositions can be formulated for topical use (e.g. as gels, creams or ointments). For instance, when used to treat (or prevent the development of) cervical cancer, the compositions can be formulated as gels, creams or ointments that may be applied directly to the cervix by techniques known to the art. Alternatively, the compositions may be formulated as a vaginal suppository (or incorporated within a pessary) according to techniques known to the art. In other aspects, the medicaments may be in the form of an ovule. The ovule can comprise a cream or gel located within a coating, the coating configured to melt and release the cream or gel upon being administered intravaginally. Alternatively, the ovule can consist of a cream or gel that is configured to melt upon being administered intravaginally. The compositions may also be formulated as a soft capsule wherein the outer layer of the capsule suitably dissolves at the site of application to allow the release of the compositions.

For oral administration, the compositions may be provided as a tablet, aqueous or oil suspension, dispersible powder or granule, emulsion, hard or soft capsule, syrup or elixir. Compositions intended for oral use may be prepared according to any method known in the art for the manufacture of pharmaceutical compositions and may include one or more of the following agents: sweeteners, flavoring agents, coloring agents and preservatives. Aqueous suspensions may contain the active ingredient in admixture with excipients suitable for the manufacture of aqueous suspensions.

Formulations for oral use may also be provided as hard gelatin capsules, wherein the active ingredient(s) are mixed with an inert solid diluent, such as calcium carbonate, calcium phosphate, or kaolin, or as soft gelatin capsules. In soft capsules, the active agents may be dissolved or suspended in suitable liquids, such as water or an oil medium, such as peanut oil, olive oil, fatty oils, liquid paraffin, or liquid polyethylene glycols. Stabilizers and microspheres formulated for oral administration may also be used. Capsules may include push-fit capsules made of gelatin, as well as soft, sealed capsules made of gelatin and a plasticizer, such as glycerol or sorbitol. The push-fit capsules may contain the active ingredient in admixture with fillers such as lactose, binders such as starches, and/or lubricants, such as talc or magnesium stearate and, optionally, stabilizers.

Tablets may be uncoated or coated by known methods to delay disintegration and absorption in the gastrointestinal tract and thereby provide a sustained action over a longer period of time. For example, a time delay material such as glyceryl monostearate may be used. When administered in solid form, such as tablet form, the solid form typically comprises from about 0.001 wt. % or less to about 50 wt. % or more of active ingredient(s), for example, from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Tablets may contain the active ingredients in admixture with non-toxic pharmaceutically acceptable excipients including inert materials. For example, a tablet may be prepared by compression or molding, optionally, with one or more additional ingredients. Compressed tablets may be prepared by compressing in a suitable machine the active ingredients in a free-flowing form such as powder or granules, optionally mixed with a binder, lubricant, inert diluent, surface active or dispersing agent. Molded tablets may be made by molding, in a suitable machine, a mixture of the powdered active agent moistened with an inert liquid diluent.

Suitable inert materials include diluents, such as carbohydrates, mannitol, lactose, anhydrous lactose, cellulose, sucrose, modified dextrans, starch, and the like, or inorganic salts such as calcium triphosphate, calcium phosphate, sodium phosphate, calcium carbonate, sodium carbonate, magnesium carbonate, and sodium chloride. Disintegrants or granulating agents may be included in the formulation, for example, starches such as corn starch, alginic acid, sodium starch glycolate, Amberlite, sodium carboxymethylcellulose, ultramylopectin, sodium alginate, gelatin, orange peel, acid carboxymethyl cellulose, natural sponge and bentonite, insoluble cationic exchange resins, powdered gums such as agar, or karaya, or alginic acid or salts thereof.

Binders may be used to form a hard tablet. Binders include materials from natural products such as acacia, starch and gelatin, methyl cellulose, ethyl cellulose, carboxymethyl cellulose, polyvinyl pyrrolidone, hydroxypropylmethyl cellulose, and the like.

Lubricants, such as stearic acid or magnesium or calcium salts thereof, polytetrafluoroethylene, liquid paraffin, vegetable oils and waxes, sodium lauryl sulfate, magnesium lauryl sulfate, polyethylene glycol, starch, talc, pyrogenic silica, hydrated silicoaluminate, and the like, may be included in tablet formulations.

Surfactants may also be employed, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

Controlled release formulations may be employed wherein the active agent or analog(s) thereof is incorporated into an inert matrix that permits release by either diffusion or leaching mechanisms. Slowly degenerating matrices may also be incorporated into the formulation. Other delivery systems may include timed release, delayed release, or sustained release delivery systems.

Coatings may be used, for example, nonenteric materials such as methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols, or enteric materials such as phthalic acid esters. Dyestuffs or pigments may be added for identification or to characterize different combinations of active agent doses.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

What is claimed is:

1. A method of treating a human papillomavirus (HPV)-related cancer in an individual in need thereof, comprising administering a glucosylceramide synthase (GCS) inhibitor to said individual.

2. The method of claim 1, wherein said cancer is selected from cervical cancer, oropharyngeal cancer, vulvar cancer, anal cancer, penile cancer, vaginal cancer, rectal cancer, squamous cell carcinoma, adenocarcinoma, and head and neck cancer.

3. The method of claim 1, wherein said HPV is a high-risk type HPV.

4. The method of claim 3, wherein said high-risk type is selected from 16, 18, 31, 33, 34, 39, 45, 51, 52, 56, 58, 59, 66, 68, 70, and combinations thereof.

5. The method of claim 1, wherein said HPV is one or both of type 16 and type 18.

6. The method of claim 1, wherein said HPV is a low-risk type HPV.

7. The method of claim 6, wherein said low-risk type is selected from 6, 11, 42, 43, and 44.

8. The method of claim 1, wherein said GCS inhibitor is selected from miglustat, eligustat, venglustat, T-036, and combinations thereof.

9. The method of claim 1, further comprising detecting an abnormal cytology in said individual, prior to said treating.

10. The method of claim 1, further comprising detecting HPV in a biological sample from said individual prior to said administering.

11. The method of claim 1, further comprising detecting a nucleic acid or a protein that indicates the presence of HPV in a biological sample from said individual prior to said administering.

12. The method of claim 11, wherein said protein is p16.

13. The method of claim 12, wherein said p16 is overexpressed as compared to a baseline or control level of p16.

14. The method of claim 11, wherein said nucleic acid indicates the presence of one or both of an E6 gene and an E7 gene.

15. The method of claim 11, wherein said biological sample is a tissue sample.

16. The method of claim 11, wherein said biological sample is from a biological sample taken from a lymph node or lesion in the head, neck, or anogenital tract.

17. The method of claim 1, wherein said individual is administered said GCS inhibitor prior to a diagnosis of said cancer.

18. The method of claim 1, wherein said individual is administered said GCS inhibitor after a diagnosis of said cancer.

19. The method of claim 1, wherein said individual is diagnosed with an HPV infection for more than one week, more than two weeks, more than three weeks, more than four weeks, more than five weeks, more than six weeks, more than seven weeks, more than two months, more than three months, more than four months, more than five months, more than six months, more than seven months, more than eight months, more than nine months, more than ten months, more than 11 months, more than 12 months, 18 months, or more than two years.

20. The method of claim 1, wherein said GCS inhibitor is administered until HPV or an indicator nucleic acid or protein thereof, is no longer detectable in a biological sample from said individual.

21. The method of claim 1, wherein said GCS inhibitor is administered for a period of time selected from about one week to about one year, or from about two weeks to about 6 months, or from about three weeks to about 3 months, or about one month.

22. The method of claim 1, wherein said administration is selected from one or both of topical and systemic.

23. The method of claim 1, wherein said HPV-related cancer is penile, anal, vulvar, cervical, and said administration is topical.

24. The method of claim 1, wherein said HPV-related cancer is head and neck cancer, and said administration is via intra-tumor injection.

25. The method of claim 1, wherein said HPV-related cancer is head and neck cancer, and said administration is systemic.

26. A method of treating an HPV-associated disease in an individual in need thereof, comprising administering a GCS inhibitor to said individual.

27. The method of claim 26, wherein said HPV-associated disease is a skin or mucosal lesion selected from a common wart, a genital wart, a respiratory wart, a plantar wart, a subungual wart, a periungual wart, a flat wart, and combinations thereof.

28. The method of claim 26, wherein said HPV type is a low-risk type HPV.

29. The method of claim 26, wherein said HPV type is one or both of type 6 and type 11.

30. The method of claim 26, wherein said administration is topical.

31. A composition comprising a GCS inhibitor, said composition being formulated for topical administration.
